(19) United States
(12) Patent Application Publication
(10) Pub. No.: US 2021/0100906 A1
(43) Pub. Date: Apr. 8, 2021

(54) METHOD FOR PRODUCING AN OIL-IN-WATER EMULSION, OIL-IN-WATER EMULSION, AND INSTALLATION FOR PRODUCING AN OIL-IN-WATER EMULSION

(71) Applicant: B. Braun Melsungen AG, Melsungen (DE)

(72) Inventors: Jürgen Schmitt, Kirchhain (DE); Volker Krüger, Nieste (DE); Carsten Grumbach, Melsungen (DE); Thorsten Braun, Niederaula (DE)

(21) Appl. No.: 17/046,394

(22) PCT Filed: Apr. 1, 2019
(86) PCT No.: PCT/EP2019/058193

§ 371 Date: Oct. 9, 2020

(30) Foreign Application Priority Data
Apr. 11, 2018 (DE) 10 2018 205 493.2

(51) Int. Cl. A61K 47/44; A61K 47/24; A61K 47/12; A61K 47/10; A61K 47/14; A61K 47/22; B01F 3/08; B01F 5/00; B01F 7/00

(52) U.S. Cl. CPC A61K 47/44; A61K 47/24; A61K 47/12; A61K 47/10; A61K 47/14; B01F 2005/0034; B01F 3/0811; B01F 3/0873; B01F 5/00; B01F 7/0075; B01F 2003/0842; A61K 47/22

(57) ABSTRACT

A system and method for producing an oil-in-water (“O/W”) emulsion performs or includes the steps of: a) providing an oil phase and a water phase, b) premixing the oil phase and the water phase to form an O/W pre-emulsion, and c) homogenizing the O/W pre-emulsion to form an O/W emulsion by at least one counter-jet disperser.

### CROSS-REFERENCE TO RELATED APPLICATIONS

This is the United States national phase entry of International Application No. PCT/EP2019/058193, filed Apr. 1, 2019, which claims the benefit of priority of German Application No. 10 2018 205 493.2, filed Apr. 11, 2018. The contents of International Application No. PCT/EP2019/058193 and German Application No. 10 2018 205 493.2 are incorporated by reference herein in their entireties.

### FIELD

The invention relates to a method for producing an oil-in-water emulsion, an oil-in-water emulsion, and a system for producing an oil-in-water emulsion.

### BACKGROUND

An emulsion is understood to mean a finely divided mixture of two normally immiscible liquids without any visible separation. One liquid (phase) forms small droplets that are distributed in the other liquid. The phase that forms droplets is called the internal or dispersed phase. The phase in which the droplets float is called the external or continuous phase. Emulsions of water and oil are differentiated into water-in-oil emulsions (W/O emulsion) and oil-in-water emulsions (O/W emulsion).

Another important constituent of emulsions is the emulsifier, which facilitates the formation of droplets and counteracts separation (phase separation).

Typically, the components used to produce an emulsion are initially pre-mixed to form a coarsely dispersed pre-emulsion, which may also be referred to as a crude or pre-emulsion or premix. This is followed by homogenization, with the dispersed phase being broken down into droplets (fine emulsification). The drop size spectrum of the crude or pre-emulsion shifts significantly towards smaller droplets.

O/W emulsions for parenteral use are usually produced by first premixing an oil phase and water phase to a pre-emulsion using a rotor-stator stirrer, followed by homogenization using a piston-gap homogenizer. Piston-gap homogenizers are so-called high-pressure homogenizers, which are based on a high-pressure pump and a homogenizing nozzle. The high-pressure pump builds up energy, which can then be used to reduce the droplet size by releasing the pressure in a homogenizing valve. In a piston-gap homogenizer, pressures from 100 to a few hundred bar can be achieved. In a piston-gap homogenizer, the pre-emulsion is generally pumped through a central feed bore, the pre-emulsion then passing through a radial gap between a valve seat and a valve piston.

In high-pressure homogenizers, shear and expansion forces, impact flows and, as is usually the case, cavitation forces are also effective to a decisive extent. Cavitation is the generation and dissolution of cavities in liquids due to pressure fluctuations. Cavitation is caused by objects moving very quickly in the liquid (for example by propellers or stirrers) or by rapid movement of the liquid, for example through a nozzle, and by exposure to ultrasound.

O/W emulsions which are intended for parenteral administration must meet certain specifications. For example, such emulsions should not exceed an average droplet diameter of 500 nm, preferably 350 nm, in order to comply with minimum medical standards.

Furthermore, such O/W emulsions should have a so-called PFAT5 value of <0.05%. This value defines the percentage of droplets within an oil phase of an O/W emulsion having a diameter, in particular a mean diameter, of 5 μm to 50 μm. This is a safety parameter to avoid fat embolism in patients.

Disadvantages of conventional processes for producing O/W emulsions are long process times and, in particular, often only limited or inadequate process control options, particularly with regard to the quality of the O/W emulsions to be produced. For instance, even slight deviations from process parameters can lead to faulty batches and consequently to batch destructions on a considerable scale.

### SUMMARY

It is an object of the invention to provide a method for producing an O/W emulsion which avoids disadvantages occurring in connection with production methods for O/W emulsions known from the prior art and which is characterized in particular by shorter process times and better process control. Objects of the invention are also to provide an associated O/W emulsion and an associated system for producing an O/W emulsion.

These objects are achieved according to the invention by a method for producing an O/W emulsion, an O/W emulsion and also by a system according to the description.

According to a first aspect, the invention relates to a method for producing an O/W emulsion, in particular for parenteral administration. The method has the following steps:
a) providing an oil phase and a water phase,
b) premixing, i.e. pre-homogenizing or pre-emulsifying, the oil phase and the water phase to form an oil-in-water pre-emulsion, i.e. an oil-in-water precursor-emulsion, hereinafter abbreviated as O/W pre-emulsion, and
c) homogenizing the O/W pre-emulsion to form an O/W emulsion by means of at least one counter-jet disperser.

In the context of the present invention, the term “water phase” is to be understood as meaning water or a water-containing liquid, in particular an aqueous solution, which in the finished O/W emulsion, i.e. in the O/W emulsion produced by the method according to the invention, forms the external or continuous phase.

In the context of the present invention, the term “oil phase” is to be understood as meaning an oil and/or lipid and/or an oil- and/or lipid-containing liquid, in particular an oil- and/or lipid-containing solution, which in the form of droplets in the finished O/W emulsion, i.e. in the O/W emulsion produced by the method according to the invention, forms the internal or dispersed phase.

In the context of the present invention, the term “droplet” is understood to mean oil droplets and/or lipid droplets, i.e. droplets consisting of at least one oil and/or at least one lipid, and/or oil- and/or lipid-containing droplets, which form the internal or dispersed phase of the O/W pre-emulsion and/or O/W emulsion. Typically in this case, the O/W pre-emulsion is characterized by a wider droplet diameter distribution and/or by droplets of larger diameter, particularly larger average diameter, than the O/W emulsion.

In the context of the present invention, the expression “counter-jet disperser” is to be understood to mean a high-pressure homogenizer in which two or more jets of a pre-emulsion (pre-emulsion or crude emulsion) from at least two, preferably two opposing, bores or channels meet each other in a droplet comminution zone. When the pre-emulsion jets meet, droplets present in the pre-emulsion are comminuted, particularly under the action of shear forces. The aforementioned droplet comminution zone can therefore also be referred to as the shear zone. The extent to which the droplets are comminuted depends in particular on the conveying speed at which the O/W pre-emulsion or O/W pre-emulsion jets are conveyed within the counter-jet disperser. The conveying speed of the O/W pre-emulsion or O/W pre-emulsion jets can be controlled via a pressure which is generated by a pump, in particular a high-pressure pump, of the counter-jet disperser.

The expression “at least one counter-jet disperser” can, as will be explained in more detail below, mean a counter-jet disperser or, which is preferred, a plurality of counter-jet dispersers, i.e. two or more counter-jet dispersers.

The at least one counter-jet disperser provided for performing step c) preferably has at least two, in particular two, preferably two opposite, channels or more channels. The channels have an internal diameter in the micrometer range, for example. As a result, particularly intensive shearing of the droplets present in the O/W pre-emulsion and consequently the production of O/W emulsions with a narrow or restricted droplet diameter distribution can be achieved.

The channels of the at least one counter-jet disperser can furthermore have, in particular, a Y-shaped configuration or arrangement.

The present invention is based, inter alia, on the following surprising findings and advantages:
by pre-emulsifying an oil phase and water phase and a subsequent high-pressure homogenization using at least one counter-jet disperser, on the one hand, the production times for O/W emulsions are significantly shortened and, on the other hand, the quality of the O/W emulsions to be produced can be better controlled and, in particular, the production can achieve qualitatively high-quality O/W emulsions.
for instance, it is possible to shorten process times by up to 75% by means of the method according to the invention. As a result, manufacturing costs can be saved to a considerable extent and the number of emulsion batches that can be produced per unit time can be increased significantly. Overall, this leads to a significant increase in productivity.
from the point of view of productivity, it is also advantageous that counter-jet dispersers generally have static, i.e. constant chamber dimensions. This facilitates the implementation of scale-up processes in that, for example, when using several counter-jet dispersers, the number of chambers can be scaled up linearly with the volume.
from a quality point of view, it is particularly advantageous that the use of at least one counter-jet disperser allows the droplet diameter distribution in the final O/W emulsion to be produced to be specified and controlled in a targeted manner. For instance, the method according to the invention particularly allows the production of O/W emulsions having a droplet diameter, in particular a mean droplet diameter (determined by photon correlation spectroscopy, PCS), of 180 nm to 340 nm, in particular 200 nm to 320 nm, preferably 200 nm to 300 nm, particularly preferably 240 nm to 280 nm.
furthermore, it is advantageous that, with the aid of the method according to the invention, as will be explained in more detail below, the PFAT5 value of the O/W emulsions to be produced can be better controlled and in particular significantly reduced. As a result, the risk of fat embolism in the case of parenteral administration of an O/W emulsion produced by means of the invention can be significantly reduced.

The water phase provided according to the invention can be provided using an emulsifier, i.e. by adding an emulsifier to water or a water-containing liquid. In particular, the water phase can be provided by dissolving an emulsifier in water or a water-containing liquid. For this purpose, the water phase can be heated to a temperature of 40° C. to 80° C., in particular 50° C. to 70° C. The emulsifier used may be a compound selected from the group consisting of phospholipids, phospholipids of animal origin, phospholipids of vegetable origin, lecithins such as egg lecithin, krill phospholipids and mixtures of at least two of the emulsifiers mentioned.

Furthermore, the water phase can be provided using an additive, i.e. by adding an additive to water or a water-containing liquid. In particular, the water phase can be provided by dissolving an additive in water or a water-containing liquid. The additive used may be a compound selected from the group consisting of emulsifying aid, stabilizer, isotonizing additive and mixture of at least two of the additives mentioned. The emulsifying aid used may be, for example, an alkali metal salt of a long-chain fatty acid, for example a fatty acid having 16 to 18 carbon atoms. The stabilizer or isotonizing additive used may be, for example, a polyhydric alcohol, particularly selected from the group consisting of glycerol, glucose, xylitol and mixtures of at least two of the stabilizers or isotonizing additives mentioned.

The oil phase can be provided using an oil and/or lipid preferably selected from the group consisting of oils of vegetable origin, medium-chain triglycerides (MCT), oils of animal origin, oils of marine origin and mixtures of at least two of the oils or lipids mentioned. The oils of vegetable origin that may be used are, for example, safflower oil and/or soybean oil. These oils are characterized by a high proportion of polyunsaturated fatty acids from the ω-6 series (predominantly linoleic acid, 18:2 ω-6), while their content of ω-3 fatty acids (almost exclusively as a-linolenic acid, 18:3 ω-3) is low. Medium-chain triglycerides (MCT) have a carbon chain length of 6 carbon atoms to 14 carbon atoms, particularly preferably 8 carbon atoms to 10 carbon atoms. The oils of marine origin that may be used are, for example, fish oils and/or krill oils. The fish oils obtained from cold water fish, like krill oils obtained from krill, are characterized by a high proportion of polyunsaturated fatty acids (mainly eicosapentaenoic acid, EPA, 20:5 ω-3 and docosahexaenoic acid, DHA, 20:6 ω-3), while their content of ω-6 fatty acids is low. Suitable fish oils, for example, are those obtained which technically consist to a significant extent of cold water fish. Fish oils generally comprise triglycerides of fatty acids having 12 to 22 carbon atoms. The fish oils that may be used are, for example, oils selected from the group consisting of sardine oil, salmon oil, herring oil, mackerel oil and mixtures of at least two of the fish oils mentioned. Alternatively or additionally, corresponding fish oil concentrates and/or krill oils can also be used.

The oil phase can also be provided using an emulsifier, i.e. by adding an emulsifier to an oil or oil mixture, to an oil-containing liquid, to a lipid or lipid mixture or to a lipid-containing liquid, especially by dissolving an emulsifier in an oil or oil mixture, an oil-containing liquid, a lipid or lipid mixture or a lipid-containing liquid. In this case, the emulsifier used can be a compound preferably selected from the group consisting of phospholipids and mixtures of at least two phospholipids.

The oil phase can also be provided using an additive, i.e. by adding an additive to an oil or oil mixture, to an oil-containing liquid, to a lipid or lipid mixture or to a lipid-containing liquid, especially by dissolving an additive in an oil or oil mixture, an oil-containing liquid, a lipid or lipid mixture or a lipid-containing liquid. The additive used may be, for example, an antioxidant, for example a tocopherol and/or physiologically harmless tocopherol ester such as a-tocopherol acetate.

Furthermore, an emulsifier-containing water phase and an emulsifier-free oil phase can be provided. This variant, referred to as the English method, for the production of an O/W emulsion has the advantage over the continental method described below that smaller amounts of emulsifier are often required.

Alternatively, an emulsifier-free water phase and an emulsifier-containing oil phase can be provided. This variant, referred to as the continental method, for the production of an O/W emulsion has the advantage that it leads to a reduction in the process time due to the better dispersibility of phospholipids.

The method according to the invention can be operated with particular advantage, in particular without modifying the process sequence and/or without converting a process plant, based either on the English method or based on the continental method.

In principle, the oil phase and the water phase can already be brought together before carrying out step b). In particular, the oil phase can already be added to the water phase before step b) is carried out.

Alternatively, the oil phase and the water phase can only be brought together when carrying out step b).

In one configuration of the invention, step b) is carried out using at least one rotor-stator disperser, in particular a rotor-stator stirrer.

The term “rotor-stator disperser” in the context of the present invention is understood to mean a disperser, in particular a stirrer or pre-homogenizer, which operates on the rotor-stator principle, i.e. comprises a rotor and a stator (so-called rotor-stator system).

The specific energy input for droplet comminution, particularly droplet shear, can be influenced with particular advantage by means of the configuration of the rotor and/or the stator of the at least one rotor-stator disperser, for example via the width and/or the number and/or the mutual spacing of shear slots, and/or via the speed of the rotor and/or via the flow rate with which the oil phase and the water phase are passed through the at least one rotor-stator-disperser.

In a further configuration of the invention, the oil phase and the water phase are fed to the at least one rotor-stator-disperser spatially separated from each another.

In a further configuration of the invention, the oil phase and the water phase are fed to the at least one rotor-stator disperser by means of a coaxial tube, i.e. a tube-in-tube arrangement, or by means of a coaxial hose, i.e. a hose-in-hose arrangement. In this way it can be ensured with particular advantage that each droplet within the at least one rotor-stator disperser is exposed to a sufficient emulsifier concentration. If the method according to the invention is operated using the English method for example, it is preferred if the oil phase is passed through the central tube or the central hose and the water phase is passed through the (coaxial) outer tube surrounding the central tube or through the (coaxial) outer hose surrounding the central hose. When using the continental method, in contrast, it is preferred if the water phase is passed through the central tube or the central hose and the oil phase is passed through the (coaxial) outer tube surrounding the central tube or through the (coaxial) outer hose surrounding the central hose. In the case of mixed forms of the methods, the configuration of the English method can be used.

In the context of the present invention, the expression “at least one rotor-stator disperser” can mean one rotor-stator disperser or a plurality of rotor-stator dispersers, i.e. two or more rotor-stator dispersers, such as two, three, four or five rotor-stator dispersers.

Accordingly, step b) can in principle be carried out using only one rotor-stator disperser.

Alternatively, step b) can be carried out using a plurality of rotor-stator dispersers, in particular using a plurality of rotor-stator dispersers connected in parallel and/or using a plurality of rotor-stator dispersers connected in series. In particular, step b) can be carried out using only rotor-stator dispersers connected in parallel. Alternatively, step b) can be carried out using only rotor-stator dispersers connected in series. With particular advantage, both the process productivity and the process quality can be increased, without affecting the process time, by connecting the rotor-stator dispersers in parallel and/or in series. The features and advantages described in the previous paragraphs in relation to the at least one rotor-stator disperser apply analogously to the use of a multiplicity of rotor-stator dispersers.

Step b) can be carried out, for example, using one or a plurality of rotor-stator dispersers commercially available under the registered trademark Inline ULTRA-TURRAX®.

In a further configuration of the invention, the oil phase and the water phase are passed, preferably exclusively, through a droplet comminution zone, in particular a shear zone, of the at least one rotor-stator disperser. In this context, the expression “droplet comminution zone” is understood to mean a zone, i.e. a region or section of the at least one rotor-stator disperser, within which droplet comminution takes place due to the action of the rotor and/or stator, particularly under the influence of shear forces. The procedure described in this paragraph can also be referred to as a forced passage of the oil phase and the water phase through the droplet comminution zone, in particular the shear zone, of the at least one rotor-stator disperser. As a result, it is particularly advantageous (already) during step b) to achieve a significant reduction in droplets which have a diameter, in particular a mean diameter, >1 μm.

In principle, the O/W pre-emulsion can be homogenized directly, i.e. without further intermediate steps, to form an O/W emulsion. As a result, a further shortening of the manufacturing time and consequently a further increase in the process productivity can be achieved in an advantageous manner.

Alternatively, the O/W pre-emulsion can be passed through an intermediate storage device or container before carrying out step c). The intermediate storage device or container, with particular advantage, serves to maintain the process flow and therefore facilitates coordination between the at least one rotor-stator disperser and the at least one counter-jet disperser.

In a further configuration of the invention, step c) is carried out using a pump pressure of 500 bar to 2000 bar, in particular 800 bar to 1900 bar, preferably 1000 bar to 1500 bar. The pump pressure disclosed in this paragraph has been found to be particularly advantageous for droplet comminution, in particular droplet shearing, and preferably for achieving a narrow or restricted droplet diameter distribution.

In the context of the present invention, the expression “pump pressure” is intended to be understood to mean a pressure generated by a pump, in particular a high-pressure pump, of the at least one counter-jet disperser. This is responsible, inter alia, for the conveying speed of the O/W pre-emulsion, in particular of O/W pre-emulsion jets, within the at least one counter-jet disperser. Therefore, the speed of impact of O/W pre-emulsion jets within a droplet comminution zone, in particular the shear zone, of the at least one counter-jet disperser and consequently the droplet comminution and thus the homogenization of the O/W pre-emulsion to form an O/W emulsion, can be controlled via the pump pressure. To this extent, the pump pressure can also be referred to as homogenizing pressure in the context of the present invention.

In a further configuration of the invention, step c) is carried out at a temperature of the O/W pre-emulsion of 30° C. to 80° C., in particular 40° C. to 77.5° C., preferably 40° C. to 75° C., particularly preferably 40° C. to 65° C. In other words, it is preferred if the at least one counter-jet disperser is operated at a temperature of the O/W pre-emulsion of 30° C. to 80° C., in particular 40° C. to 77.5° C., preferably 40° C. to 75° C., particularly preferably 40° C. to 65° C., or, to put it another way, the O/W pre-emulsion when carrying out step c) has a temperature of 30° C. to 80° C., in particular 40° C. to 77.5° C., preferably 40° C. to 75° C., particularly preferably 40° C. to 65° C. The temperature disclosed in this paragraph can therefore also be referred to as the homogenization temperature in the context of the present invention. The temperature disclosed in this paragraph has (also) been found to be particularly advantageous for droplet comminution, in particular droplet shearing, and preferably for achieving a narrow or restricted droplet diameter distribution.

For example, the at least one counter-jet disperser for carrying out step c) can be operated at a pump pressure of 1900 bar and at a temperature of the O/W pre-emulsion of 40° C.

The at least one counter-jet disperser for carrying out step c) can also be operated, for example, at a pump pressure of 1500 bar and at a temperature of the O/W pre-emulsion of 50° C.

The at least one counter-jet disperser for carrying out step c) can also be operated, for example, at a pump pressure of 1000 bar and at a temperature of the O/W pre-emulsion of 60° C.

In a further configuration of the invention, the O/W pre-emulsion is passed two or more times, in particular twice, three times, four times or five times through the at least one counter-jet disperser when carrying out step c). By passing the O/W pre-emulsion through the at least one counter-jet disperser several times, an increase in the process quality, in particular with regard to the mean droplet diameter and/or the PFAT5 value, can be achieved with particular advantage.

In a further configuration of the invention, step c) is carried out by means of a plurality of counter-jet dispersers, in particular by means of two, three, four or five counter-jet dispersers. Step c) is preferably carried out by means of a plurality of counter-jet dispersers connected in parallel and/or by means of a plurality of counter-jet dispersers connected in series. By using a plurality of counter-jet dispersers, in particular by connecting the counter-jet dispersers in parallel and/or in series, a significant improvement in process quality can also be achieved, in particular with regard to the mean droplet diameter and/or the PFAT5 value. In addition, this procedural measure(s) can increase process productivity. The process time does not change if the counter jet dispersers are connected in series. In the case of a parallel connection, the process time is reduced linearly. Overall, there is therefore a considerable time saving, whereby a significant increase in the number of O/W emulsion batches that can be produced per unit time and consequently a significant increase in process productivity can be achieved.

In particular, step c) can be carried out using only counter-jet dispersers connected in parallel.

Alternatively, step c) can be carried out using only counter-jet dispersers connected in series.

In a further configuration of the invention, step c) is carried out using at least two counter-jet dispersers connected in series, in particular using only two counter-jet dispersers connected in series. The first counter-jet disperser is preferably operated at a higher pump pressure than the second, i.e. downstream, counter-jet disperser. The first counter-jet disperser is particularly preferably operated at a pump pressure of at most 1900 bar, preferably at most 1500 bar, in particular at a pump pressure of 800 bar to 1400 bar, preferably 1000 bar to 1200 bar, and the second, i.e. downstream, counter-jet disperser is operated at a pump pressure <1000 bar, in particular from 500 bar to 800 bar, preferably 500 bar.

The present invention is further based on the surprising finding that droplets having a diameter, particularly a mean diameter, of 1 μm to 50 μm may be comminuted with preference at a pump pressure of <1000 bar, in particular from 500 bar to 800 bar, preferably 500 bar. As a result, with particular advantage, the PFAT5 value can be controlled, which is important for O/W emulsions to be administered parenterally.

Furthermore, the present invention is based on the surprising finding that by means of a pressure cascade, in particular by means of at least two counter-jet dispersers connected in series, wherein—as described in the penultimate paragraph—the first counter-jet disperser is operated at a higher pump pressure than the second (downstream) counter-jet disperser, droplets can be produced having a diameter, in particular a mean diameter, <500 nm, in particular <400 nm, preferably <350 nm, in particular from 200 nm to 320 nm, preferably 200 nm to 300 nm, particularly preferably 240 nm to 280 nm, specifically by means of the pump pressure of the first counter-jet disperser, and in addition the proportion of droplets having a diameter, in particular a mean diameter, ≥1 μm, in particular from 1 μm to 50 μm, can be reduced significantly and in particular reproducibly, specifically by means of the pump pressure of the second counter-jet disperser.

For example, the first counter-jet disperser can be operated at a pump pressure of 1900 bar and the second counter-jet disperser at a pump pressure of 500 bar.

Also by way of example, the first counter-jet disperser can be operated at a pump pressure of 1500 bar and the second counter-jet disperser at a pump pressure of 500 bar.

Also by way of example, the first counter-jet disperser can be operated at a pump pressure of 1200 bar and the second counter-jet disperser at a pump pressure of 500 bar.

Furthermore, the first counter-jet disperser and the second counter-jet disperser can each be operated at the same temperature of the O/W pre-emulsion. In this respect, reference is made to the homogenization temperatures already disclosed in the description so far in connection with the O/W pre-emulsion. For example, both the first counter-jet disperser and the second counter-jet disperser can be operated at a temperature of the O/W pre-emulsion of 50° C.

As counter-jet disperser, it is possible to use one or a plurality of counter-jet dispersers available commercially under the tradename Nanojet or registered trademark Microfluidizer®.

In a further configuration, a pressure reducer is connected downstream of the at least one counter-jet disperser. The pressure reducer is preferably configured to generate a counter pressure to a pressure, in particular a pump pressure, generated by the at least one counter-jet disperser. For example, the pressure reducer can be configured to generate a counter pressure of 10 bar to 100 bar, in particular 30 bar to 70 bar. By using a pressure reducer, process stability can be achieved with particular advantage. In particular, outgassing phenomena can be avoided by means of a pressure reducer. In the case of a plurality of counter-jet dispersers, a pressure reducer can be connected downstream of each counter-jet disperser.

The method according to the invention is preferably used to produce an O/W emulsion having a droplet diameter, in particular a mean droplet diameter (determined by photon correlation spectroscopy, PCS) of 180 nm to 340 nm, in particular 200 nm to 320 nm, preferably 240 nm to 280 nm.

Furthermore, the method according to the invention is used to produce an O/W emulsion having a PFAT5 value <0.05%, in particular <0.04%, preferably <0.03%, more preferably <0.02%, particularly preferably 0.01%, especially <0.01%. For example, an O/W emulsion having a PFAT5 value of 0.001% to 0.01% can be produced by the method according to the invention.

In a further configuration of the invention, an O/W emulsion to be administered parenterally is produced by the method according to the invention.

According to a second aspect, the invention relates to an oil-in-water emulsion, hereinafter abbreviated as O/W emulsion, which is produced or can be produced by a method according to the first aspect of the invention.

Alternatively or in combination, in accordance with a second aspect, the invention relates to an oil-in-water emulsion, hereinafter abbreviated as O/W emulsion, having a PFAT5 value <0.04%, in particular <0.03%, preferably <0.02%, particularly preferably 0.01%, especially <0.01%. For example, the O/W emulsion can have a PFAT5 value of 0.001% to 0.01%.

It is also preferred if the O/W emulsion has a droplet diameter, in particular a mean droplet diameter (determined by photon correlation spectroscopy, PCS), of 180 nm to 340 nm, in particular 200 nm to 320 nm, preferably 200 nm to 300 nm, particularly preferably 240 nm to 280 nm.

With regard to further features and advantages of the O/W emulsion, reference is made in full to the statements made in the context of the first aspect of the invention. The features and advantages described therein in connection with the method according to the invention also apply analogously to the O/W emulsion according to the second aspect of the invention.

According to a third aspect, the invention relates to a system for producing an oil-in-water emulsion, hereinafter abbreviated as O/W emulsion, and/or for carrying out a method according to the first aspect of the invention.

The system has at least one disperser for pre-mixing, i.e. pre-homogenizing or pre-emulsifying an oil phase and a water phase to form an oil-in-water pre-emulsion (oil-in-water precursor-emulsion), hereinafter abbreviated as O/W pre-emulsion, and at least one preferably downstream counter-jet disperser for homogenizing the O/W pre-emulsion to an oil-in-water emulsion, hereinafter abbreviated as O/W emulsion.

The at least one disperser (for premixing the O/W pre-emulsion) is preferably designed as a rotor-stator disperser, in particular a rotor-stator.

The system may have one rotor-stator disperser or a plurality of rotor-stator dispersers, i.e. two or more rotor-stator dispersers, such as two, three, four or five rotor-stator dispersers, for example.

In particular, the system may have a plurality of rotor-stator dispersers connected in parallel and/or a plurality of rotor-stator dispersers connected in series.

Furthermore, the system may have one counter-jet disperser or a plurality of counter-jet dispersers, i.e. two or more counter-jet dispersers, such as two, three, four or five counter-jet dispersers, for example.

In particular, the system may have a plurality of counter-jet dispersers connected in parallel and/or a plurality of counter-jet dispersers connected in series.

The system preferably has at least two counter-jet dispersers connected in series.

Furthermore, an intermediate container may be connected between the at least one disperser (for premixing the O/W pre-emulsion) and the at least one counter-jet disperser. The intermediate container facilitates the coordination between the at least one rotor-stator disperser and the at least one counter-jet disperser with particular advantage by buffering the process flow.

Furthermore, a pressure reducer may be connected downstream of the at least one counter-jet disperser. Further features and advantages of the invention arise from the following description of preferred embodiments in the form of figure descriptions and the associated figures as well as examples. In this case, single features can each be implemented individually or in combination with one another. The embodiments described below merely reflect the present invention by way of example without restricting it thereto.

### BRIEF DESCRIPTION OF THE DRAWING FIGURES

The following is shown schematically in the figures:

FIG. 1 is a schematic view of a microchannel structure of a counter-jet disperser which can be used according to the invention;

FIG. 2 is a flow diagram of a method according to the invention;

FIG. 3 is a further flow diagram of a method according to the invention; and

FIG. 4 is a further flow diagram of a method according to the invention.

### DETAILED DESCRIPTION

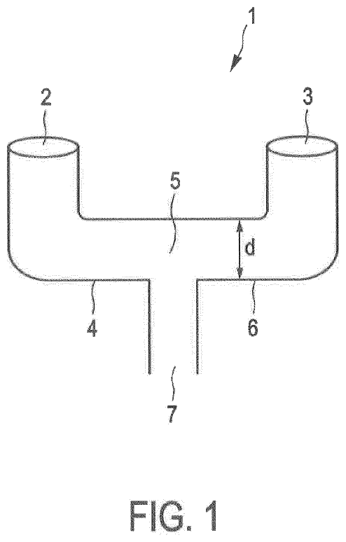

FIG. 1 shows a channel structure 1 of a counter-jet disperser which can be used according to the invention. The channel structure 1 shown has a Y-shaped arrangement and can, for example, have an internal diameter d in the micrometer range. An O/W pre-emulsion can be passed through the channel structure 1 via the inlets 2 and 3 by means of a pressure generated by a pump (high-pressure pump) of the counter-jet disperser. Due to the oppositely arranged channels 4 and 6, jets of the O/W pre-emulsion encounter each other in a droplet comminution zone 5. Particularly under the influence of shear forces, there is a comminution of the droplets present in the O/W pre-emulsion. The resulting O/W emulsion can exit the channel structure 1 via an outlet 7.

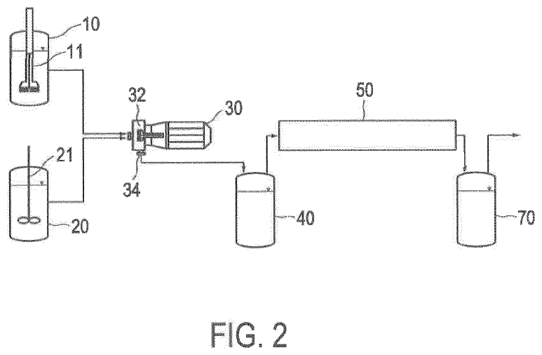

FIG. 2 shows schematically a flow diagram of a method according to the invention according to the English method.

A pre-disperser 10 with a rotor-stator system 11 is used to provide a water phase. This enables an emulsifier, such as egg lecithin, to be dispersed in water, particularly in water for injection purposes (WFI). In addition to an emulsifier, the water can also be mixed with a stabilizer or isotonizing agent, such as glycerol, and with an emulsifying aid, such as sodium oleate. Subsequently, the mixture can be heated or temperature-controlled, for example to a temperature of 55° C. to 75° C., over a period of 60 minutes.

An oil phase can be provided in a container 20, which can be configured as a pre-temperature control container, with a stirring element 21. For example, soybean oil and medium-chain triglycerides (MCT) as well as a-tocopherol can be used to provide the oil phase. The mixture produced in the container 20 can also be heated or temperature-controlled, for example to a temperature of 55° C. to 75° C.

The oil phase and water phase provided in this manner are then fed into a rotor-stator disperser 30. In this case, the oil phase and the water phase are preferably fed into the rotor-stator disperser 30 spatially separated from each other. This can be effected, for example, by means of a coaxial tube or a coaxial hose. This ensures that the oil droplets are exposed to a sufficient emulsifier concentration.

The oil phase and the water phase are preferably passed through a shear zone 32 of the rotor-stator disperser 30. As a result, an effective comminution of oil droplets with a diameter, in particular a mean diameter, ≥1 μm can already be achieved at this process stage. In the rotor-stator disperser 30, the oil phase and the water phase are premixed to form an O/W pre-emulsion.

Via an outlet 34 of the rotor-stator disperser, the O/W pre-emulsion can be fed to at least one counter-jet disperser 50 via an intermediate storage container 40. The intermediate storage device or container, with particular advantage, serves to maintain the process flow and therefore facilitates coordination between the rotor-stator disperser 30 and the at least one counter-jet disperser 50.

The counter-jet disperser 50 is operated by means of a high-pressure pump which, in particular, can generate a pressure in the range from 500 bar to 1900 bar. By means of the pump pressure generated within the counter-jet disperser 50, the O/W pre-emulsion is pumped through a microchannel structure with preferably opposing channels. In this case, jets of the O/W pre-emulsion meet each other in the droplet comminution zone, as a result of which droplets present in the O/W pre-emulsion are comminuted, in particular under the effect of shear forces. In this case, with particular advantage, droplets can be generated having a diameter, in particular a mean diameter (determined by photon correlation spectroscopy, PCS), of 180 nm to 340 nm, in particular 200 nm to 320 nm, preferably 240 nm to 280 nm.

The O/W emulsion generated in the counter-jet disperser 50 can then be transferred to a filling container 70 for further filling in suitable packaging sizes.

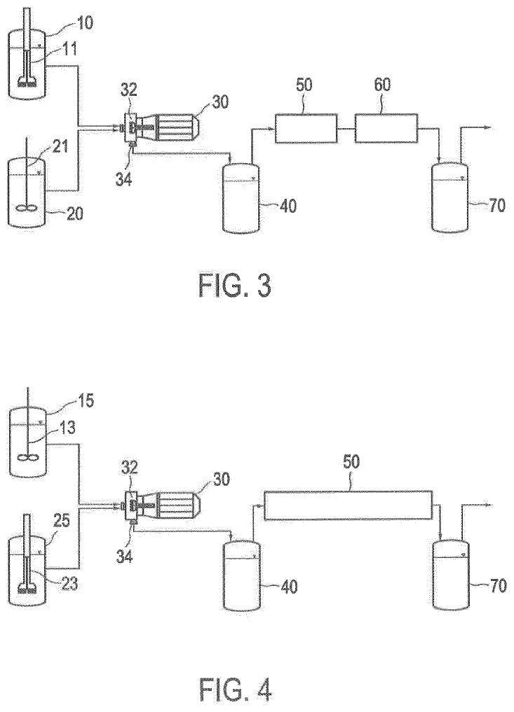

FIG. 3 shows schematically a further flow diagram of a method according to the invention which is operated according to the English method.

The method shown differs from the method shown in FIG. 1 in that it is operated with two counter-jet dispersers 50 and 60 connected in series.

In this case, droplets having a diameter, in particular a mean diameter (determined by photon correlation spectroscopy, PCS), of 180 nm to 340 nm, in particular 200 nm to 320 nm, preferably 200 nm to 300 nm, particularly preferably 240 nm to 280 nm, are preferably generated in the first counter-jet disperser 50, while in the second, i.e. downstream, counter-jet disperser 60, there is preferably a reduction in the proportion of droplets having a diameter, in particular a mean diameter, of ≥1 μm and therefore a reduction in the PFAT5 value. For this purpose, the first counter-jet disperser 50 can be operated, for example, at a pump pressure of 1500 bar, the O/W pre-emulsion within the counter-jet disperser 50 preferably having a temperature of 50° C. The second counter-jet disperser 60 is preferably operated at a pump pressure of 500 bar, the O/W emulsion within the counter-jet disperser 60 preferably having a temperature of 50° C.

Otherwise, the process sequence and the reference numbers correspond to the process sequence shown in FIG. 2 and to the reference numbers shown in FIG. 2.

FIG. 4 shows schematically a further flow diagram of a method according to the invention. In this case, however, the procedure is based on the continental method.

For this purpose, a water phase is provided by means of a container 15, which can be configured as a pre-temperature control container, and the oil phase is provided by means of a pre-disperser 25 with a rotor-stator system 23.

To provide the water phase, water, in particular water for injection purposes (WFI), can be admixed, for example, with aqueous sodium hydroxide solution and glycerol and the mixture thus obtained can be heated or temperature-controlled, for example, to a temperature of 55° C. to 75° C. with stirring by means of a stirrer element 13. To provide the oil phase, for example, oleic acid, soybean oil and medium-chain triglycerides can be admixed with an emulsifier, such as egg lecithin, and an antioxidant, such as a-tocopherol, and the mixture thus obtained can also be heated or temperature-controlled with stirring to a temperature of 55° C. to 75° C.

Otherwise, the process sequence and the reference numbers correspond to the process sequence shown in FIG. 2 and to the reference numbers shown in FIG. 2.

### EXAMPLE SECTION

1. Preparation of a Parenteral Fat Emulsion (Lipofundin MCT/LCT 20%)

The preparation process was divided into the following three process steps.

In a first step, the oil phase and water phase were prepared. The water phase was prepared in a stirred tank reactor for comminuting and dissolving the emulsifier. The oil phase was produced by simply controlling the temperature of the oil phase on a magnetic stirrer.

In a second step, an O/W pre-emulsion was prepared by means of a rotor-stator disperser commercially available under the registered trademark Inline ULTRA-TURRAX® (Ytron-Z). In contrast to conventional processes, the oil phase and the water phase were passed through the shear zone of the rotor-stator disperser by means of a forced passage. This ensured that every part of the oil phase also passed through the homogenization zone. In classical stirred tank reactors, the introduction of the oil phase into the rotor-stator stirrer can only be considered statistically and experience has shown that it leads to an undesirably broad particle distribution which can only be controlled to a limited extent.

In a third step, the final fine emulsion was produced using a high pressure homogenizer of the PSI-40 type configured as a counter-jet disperser. In contrast to the piston-gap homogenizers used in conventional processes, which use a dynamic valve to break up the droplets, the counter-jet disperser has a static microchannel structure for breaking up the droplets.

1.1 Rotor-Stator Disperser (Inline Rotor-Stator, Ytron-Z)

The rotor-stator disperser (Ytron-Z) used consisted of eleven main components. The raw materials (oil phase and water phase) could be fed for metered addition to the system via two feed funnels, which could each be closed or opened via a disk valve. From there, the raw materials ran directly into the inlet of two diaphragm motor-driven metering pumps (Sigma/i Control Type S1Cb available under the registered trademark ProMinent®). These two pumps worked on the principle of an oscillating displacement pump, which was driven by an electric motor. This transmitted a stroke movement to a metering diaphragm by means of a push rod. The stroke movement of the displacer was continuously recorded and regulated, so that the stroke could be carried out according to a predefined metering profile and thus could be adapted accordingly to the properties of the raw materials (viscosity and/or outgassing property). So that each oil droplet was exposed to a direct emulsifier concentration, the metered addition was conducted via a metering head having a tube-in-tube structure. While the oil phase was fed through the center of the inner tube, the water phase was fed into a surrounding outer tube. The raw materials were pumped directly into a reactor head by means of the two metering pumps and there ran through a forced passage directly into a rotating rotor/stator set. This was driven by a three-phase motor (ATB Motorenwerke GmbH, IM B3; 1.5 kW).

The product passed the rotor/stator and left the reactor head via a product outlet which was narrowed by a compressed air-driven pinch valve (KVT GmbH).

The pinch valve served on the one hand as a technically obligatory counter pressure valve for the correct functionality of the two diaphragm metering pumps, on the other hand as a reduction unit for the product outlet to guarantee that the reactor head reached its working volume and could not run empty during the process. The system was controlled via a switch cabinet using a programmable logic controller (PLC, SIMATIC, Siemens AG). The ratios of the two metering pumps and the speed of the rotor-stator disperser could be entered and started simultaneously via a touch panel mounted on the switch cabinet door. The shaft of the rotor-stator disperser was sealed by means of a product-lubricated mechanical ring seal.

The rotor disk was clamped onto the rotary shaft of the three-phase motor by means of a feather key and was firmly fixed to it by means of a rotor screw with an O-ring seal. The stator was firmly screwed onto the reactor cover and was moved without contact against the rotor disk when the reactor head was closed. The reactor head was closed using a clamp connection with an O-ring seal.

1.2 Formulation of a Model Emulsion (Lipofundin MCT/LCT 20%; Parenteral Fat Emulsion)

To prepare an example of an O/W emulsion, the formulation given in Table 1 below was used:

TABLE 1
Formulation of a model emulsion (parenteral fat emulsion)

| Material | Amount [g] |
|---|---|
| Egg lecithin | 120 |
| Sodium oleate | 3 |
| Glycerol | 250 |
| Soybean oil | 1000 |
| Medium chain triglycerides (MCT) | 1000 |
| alpha-tocopherol | 2 |
| Water for injection purposes | filled up to 10 l |

1.3 Procedure:

The water phase was produced in a 10 l stirred tank, which was heated to a process temperature of 65° C. by means of a temperature control unit via a double jacket.

This process step essentially served to comminute and hydrate the emulsifier in the water phase. For this process step, egg lecithin (emulsifier), glycerol and sodium oleate were placed in a stirred tank and made up to a volume of 10 l with temperature-controlled (65° C.) water for injection purposes (WfI).

For the dispersion, a rotor-stator stirrer, obtainable under the registered trademark IKA T 50 ULTRA-TURRAX®, was used at maximum speed (10,000 rev/min). The dispersion was effected for 1 h in the stirred tank on the rotor-stator stirrer with simultaneous temperature control at 65° C. by means of the jacket temperature control of the stirred tank.

Subsequently, the water phase was further temperature-controlled at a process temperature of 75° C. on a magnetic stirrer, in preparation for use in the disperser, and transferred to a first storage container of the inline rotor-stator reactor. This also had a jacket temperature control which brought the water phase to the process temperature during the emulsification. The preparation of the water phase was completed with this step.

To produce the oil phase, soybean oil, MCT and alpha-tocopherol were placed in a glass beaker and then temperature-controlled at a process temperature of 75° C. on a magnetic stirrer, in preparation for use in the disperser, and transferred to a second storage container of the inline rotor-stator reactor. This storage container also had a jacket temperature control which brought the oil phase to the process temperature during the emulsification. The preparation of the oil phase was completed with this step.

A rotor with a slot width of 1 mm and a stirrer circumference of 33 mm for an innermost toothed ring, a stirrer circumference of 44 mm for a central toothed ring and a stirrer circumference of 55 mm for an outer toothed ring was used.

The tooth spacing of the stator was 0.5 mm. The circumference of the three toothed rings was 38 mm for an inner toothed ring, 49 mm for a middle toothed ring and 60 mm for an outer toothed ring.

Prior to the start of the emulsification, the process parameters for the metered addition and for the rotor-stator speed were set on the PLC of the control unit of the inline rotor-stator according to the following Table 2:

TABLE 2
Process parameters of the inline rotor-stator

| Process parameters | Setting | |
|---|---|---|
| Speed | 5860 | rpm |
| Metered addition | 75 | L/h |
| Counter pressure | 2 | bar |
| Temperature | 75° | C. |

After starting the system, the pressure at the product outlet was set to a counter pressure of 2 bar. The O/W pre-emulsion was collected at the product outlet in a glass beaker and continuously maintained under stirring.

The O/W emulsion was then finely emulsified by three passes in a high-pressure homogenizer of the PSI-40 type configured as a counter-jet disperser. Instead of a conventional dynamic valve, this high-pressure homogenizer used a static micrometer-sized channel structure in which the droplet breakup took place. Due to the much narrower and invariant channel dimensions, there was more intensive shear and a lower and reproducible flow distribution with resulting narrow droplet distributions. In addition, due to their static chamber geometry, such high pressure homogenizers can be scaled more easily. The droplet break-up took place in an interaction chamber (shear chamber), consisting of a diamond core which was sunk into a 316L stainless steel casing. The diamond core was provided with the microstructured channels mentioned above, in which the droplets were accelerated and broken up at a high process pressure. So-called Y-chambers were used for the emulsification. The microchannels in such chambers were formed into a Y-shape. In this case, process pressures of 500 bar to 2000 bar were possible.

In order to protect the interaction chamber from damage caused by cavitation at high process pressures, an APM (auxiliary processing module) was connected downstream of the interaction chamber (secondary chamber). This secondary chamber functioned as a pressure reducer and generated a low counter pressure on the outlet side (outlet) of the primary chamber. Depressurization of the interaction chamber against the direct atmospheric pressure with induced cavitation was thus prevented. In practice, the APM module was a stainless steel core provided with a specially dimensioned hole in a stainless steel casing.

The following process parameters and chamber configurations were established for the high pressure homogenizer:

TABLE 3
Process parameters and chamber configurations
of the high pressure homogenizer

| Process parameters | Setting | |
|---|---|---|
| Pressure | 1000 | bar |
| Temperature | 60° | C. |

| | |
|---|---|
| Passes | 3 |
| Primary chamber | E101D |
| (interaction chamber) | |
| Secondary chamber (APM) | APM |

The E101D chamber was a single-slot Y-chamber and provided flow rates of up to 20 L/h.

The APM module provided a counter pressure of ca. 50 bar for the primary chamber E101D.

By further optimization of the chamber configuration, an additionally improved emulsion quality could be achieved with the PSI-40 high pressure homogenizer. This configuration was set with the following process parameters:

TABLE 4
further optimized process parameters and chamber
configurations of the high pressure homogenizer

| Process parameters | Setting | |
|---|---|---|
| Pressure | 1000 | bar |
| Temperature | 60° | C. |

| | |
|---|---|
| Passes | 3 |
| Primary chamber | E101D |
| (interaction chamber) | |
| Secondary chamber (APM) | APM (reduced counter |
| | pressure) |

The E101D chamber was a single-slot Y-chamber and provided flow rates of up to 20 L/h.

The APM module (reduced counter pressure) provided a counter pressure for the primary chamber E101D, but with a reduced counter pressure close to 50 bar.

Information regarding the counter pressures generated was based on the manufacturer's data.

The following particle analysis was used to characterize the O/W emulsions produced.

a) Photon Correlation Spectroscopy (PCS):

Using this method, Brownian molecular motion is quantified with the aid of an autocorrelation function of the scattered light signal of dispersed particles. For the measurement, a light beam of a defined wavelength is passed through a sample by means of a laser, whereby the laser light is scattered. The scattered light intensity is subject to time-dependent fluctuations due to the undirected diffusion of molecules which surround the particles. These time-dependent interference phenomena are dependent on the size of the scattering particles.

The mean particle or droplet diameter in nanometers [nm] is used as the output parameter.

b) Microscopic Image Recording (Micrograph):

For the microscopic image recording, one droplet (ca. 10 l sample) in each case was viewed on a slide under a light microscope with a ×100 immersion oil lens. A sample image was taken from this sample at five points (top left, bottom left, bottom right, top right, center) on the slide, which was then evaluated using software by counting droplets over a size of 2 μm.

The micrograph with the unit [droplet] was used as output parameter. The micrograph corresponded to the number of droplets from five sample images of one observed sample volume.

2. Preparation of O/W Emulsions at Different Homogenization Temperatures and Pressures

The fat emulsion prepared according to 1. was prepared using different homogenization temperatures and pressures. A type PSI-40 counter-jet disperser was used. From a description by Microfluidics (Chamber User Guide, Dec. 30, 2014), it is known how the process temperature changes with pressure during the homogenization (2.5° C. per 100 bar). This temperature is to be added to the respective test temperature T_{H} of the O/W pre-emulsion, i.e. the temperature of the O/W pre-emulsion before it enters the at least one counter-jet disperser, and gives the homogenization temperature in the context of the present invention. For example, for an O/W pre-emulsion having a temperature of 20° C. before it enters the at least one counter-jet disperser, a temperature of the O/W pre-emulsion within the counter-jet disperser of 45° C. is calculated in the case of a counter-jet disperser which is operated at a homogenizing pressure (pump pressure) of 1000 bar.

Measured were the percentage of emulsion droplets larger than 5 micrometers (PFAT5), the mean particle or droplet diameter (MDS=mean droplet size), measured using photon correlation spectroscopy (PCS), the number of droplets using microscopic counting, and the pH value.

2.1 20° C. Study

TABLE 5
Investigation of emulsion parameters
after preparation of a fat emulsion at 20° C.
(test temperature) and different homogenization pressures
T_{H} = 20° C.

| | | | | Mean | Micro- | |
|---|---|---|---|---|---|---|
| | Theoretical | | | particle | graph | |
| Pressure | temperature | | | diameter | [Number | |
| P | increase | Passes | pFat5 | (PCS) | of | |
| [bar] | [° C.] | PSI-40 | [%] | [nm] | droplets] | pH |
| 1900 | 47.5 | 1 | 0.030 | 280 | 46 | 8.27 |
| 1900 | 47.5 | 2 | 0.011 | 237 | 18 | 8.19 |
| 1900 | 47.5 | 3 | 0.004 | 222 | 13 | 8.16 |
| 1900 | 47.5 | 4 | 0.009 | 209 | 14 | 8.12 |
| 1900 | 47.5 | 5 | 0.003 | 205 | 7 | 8.08 |
| 1500 | 37.5 | 1 | 0.027 | 307 | 38 | 8.24 |
| 1500 | 37.5 | 2 | 0.005 | 260 | 17 | 8.17 |
| 1500 | 37.5 | 3 | 0.003 | 242 | 12 | 8.14 |
| 1500 | 37.5 | 4 | 0.002 | 234 | 9 | 8.10 |
| 1500 | 37.5 | 5 | 0.002 | 223 | 5 | 8.07 |
| 1000 | 25.0 | 1 | 0.030 | 348 | 28 | 8.24 |
| 1000 | 25.0 | 2 | 0.010 | 296 | 14 | 8.09 |
| 1000 | 25.0 | 3 | 0.002 | 269 | 12 | 8.13 |
| 1000 | 25.0 | 4 | 0.001 | 260 | 9 | 8.10 |
| 1000 | 25.0 | 5 | 0.001 | 263 | 6 | 8.07 |
| 500 | 12.5 | 1 | 0.039 | 434 | 26 | 8.29 |
| 500 | 12.5 | 2 | 0.004 | 354 | 14 | 8.20 |
| 500 | 12.5 | 3 | 0.007 | 347 | 5 | 8.15 |
| 500 | 12.5 | 4 | 0.002 | 327 | 7 | 8.14 |
| 500 | 12.5 | 5 | 0.002 | 321 | 5 | 8.11 |

2.2 30° C. Study

TABLE 6
Investigation of emulsion parameters
after preparation of a fat emulsion at 30° C.
(test temperature) and different homogenization pressures
T_{H} = 30° C.

| | | | | Mean | Micro- | |
|---|---|---|---|---|---|---|
| | Theoretical | | | particle | graph | |
| | temperature | | | diameter | [Number | |
| Pressure | increase | Passes | pFat5 | (PCS) | of | |
| [bar] | [° C.] | PSI-40 | [%] | [nm] | droplets] | pH |
| 1900 | 47.5 | 1 | 0.033 | 273 | 39 | 7.88 |
| 1900 | 47.5 | 2 | 0.011 | 233 | 19 | 7.78 |
| 1900 | 47.5 | 3 | 0.006 | 219 | 20 | 7.74 |
| 1900 | 47.5 | 4 | 0.003 | 209 | 17 | 7.72 |
| 1900 | 47.5 | 5 | 0.004 | 202 | 16 | 7.70 |
| 1500 | 37.5 | 1 | 0.021 | 295 | 33 | 7.84 |
| 1500 | 37.5 | 2 | 0.004 | 252 | 16 | 7.69 |
| 1500 | 37.5 | 3 | 0.002 | 235 | 15 | 7.69 |
| 1500 | 37.5 | 4 | 0.001 | 226 | 14 | 7.85 |
| 1500 | 37.5 | 5 | 0.001 | 217 | 8 | 7.63 |
| 1000 | 25.0 | 1 | 0.029 | 360 | 21 | 7.85 |
| 1000 | 25.0 | 2 | 0.003 | 286 | 12 | 7.79 |
| 1000 | 25.0 | 3 | 0.004 | 258 | 17 | 7.75 |
| 1000 | 25.0 | 4 | 0.001 | 252 | 7 | 7.72 |
| 1000 | 25.0 | 5 | 0.001 | 246 | 8 | 7.70 |
| 500 | 12.5 | 1 | 0.042 | 430 | 26 | 7.87 |
| 500 | 12.5 | 2 | 0.006 | 353 | 8 | 7.81 |
| 500 | 12.5 | 3 | 0.009 | 336 | 7 | 7.78 |
| 500 | 12.5 | 4 | 0.005 | 324 | 3 | 7.75 |
| 500 | 12.5 | 5 | 0.007 | 307 | 6 | 7.69 |

2.3 40° C. Study

TABLE 7
Investigation of emulsion parameters
after preparation of an emulsion at 40° C.
(test temperature) and different homogenization pressures
T_{H} = 40° C.

| | | | | Mean | Micro- | |
|---|---|---|---|---|---|---|
| | Theoretical | | | particle | graph | |
| | temperature | | | diameter | [number | |
| Pressure | increase | Passes | pFat5 | (PCS) | of | |
| [bar] | [° C.] | PSI-40 | [%] | [nm] | droplets] | pH |
| 1900 | 47.5 | 1 | 0.099 | 257 | 43 | 8.31 |
| 1900 | 47.5 | 2 | 0.022 | 221 | 26 | 8.23 |
| 1900 | 47.5 | 3 | 0.013 | 209 | 19 | 8.17 |
| 1900 | 47.5 | 4 | 0.008 | 204 | 12 | 8.15 |
| 1900 | 47.5 | 5 | 0.006 | 200 | 12 | 8.15 |
| 1500 | 37.5 | 1 | 0.045 | 278 | 58 | 8.29 |
| 1500 | 37.5 | 2 | 0.013 | 239 | 19 | 8.20 |
| 1500 | 37.5 | 3 | 0.005 | 227 | 12 | 8.18 |
| 1500 | 37.5 | 4 | 0.003 | 211 | 7 | 8.12 |
| 1500 | 37.5 | 5 | 0.002 | 209 | 13 | 8.12 |
| 1000 | 25.0 | 1 | 0.028 | 315 | 35 | 8.29 |
| 1000 | 25.0 | 2 | 0.004 | 265 | 17 | 8.19 |
| 1000 | 25.0 | 3 | 0.004 | 241 | 7 | 8.12 |
| 1000 | 25.0 | 4 | 0.001 | 231 | 9 | 8.09 |
| 1000 | 25.0 | 5 | 0.002 | 227 | 5 | 8.09 |
| 500 | 12.5 | 1 | 0.043 | 381 | 21 | 8.33 |
| 500 | 12.5 | 2 | 0.003 | 339 | 7 | 8.24 |
| 500 | 12.5 | 3 | 0.003 | 309 | 4 | 8.20 |
| 500 | 12.5 | 4 | 0.002 | 294 | 6 | 8.17 |
| 500 | 12.5 | 5 | 0.001 | 278 | 2 | 8.13 |

2.4 50° C. Study

TABLE 8
Investigation of emulsion parameters
after preparation of an emulsion at 50° C.
(test temperature) and different homogenization pressures
T_{H} = 50° C.

| | | | | Mean | Micro- | |
|---|---|---|---|---|---|---|
| | Theoretical | | | particle | graph | |
| | temperature | | | diameter | [Number | |
| Pressure | increase | Passes | pFat5 | (PCS) | of | |
| [bar] | [° C.] | PSI-40 | [%] | [nm] | droplets] | pH |
| 1900 | 47.5 | 1 | 0.102 | 257 | 66 | 8.31 |
| 1900 | 47.5 | 2 | 0.039 | 221 | 29 | 8.24 |
| 1900 | 47.5 | 3 | 0.020 | 201 | 35 | 8.22 |
| 1900 | 47.5 | 4 | 0.021 | 291 | 34 | 8.18 |
| 1900 | 47.5 | 5 | 0.021 | 197 | 35 | 8.18 |
| 1500 | 37.5 | 1 | 0.072 | 266 | 41 | 8.30 |
| 1500 | 37.5 | 2 | 0.016 | 231 | 26 | 8.20 |
| 1500 | 37.5 | 3 | 0.009 | 216 | 21 | 8.15 |
| 1500 | 37.5 | 4 | 0.005 | 205 | 13 | 8.16 |
| 1500 | 37.5 | 5 | 0.006 | 199 | 9 | 8.13 |
| 1000 | 25.0 | 1 | 0.036 | 293 | 22 | 8.35 |
| 1000 | 25.0 | 2 | 0.005 | 253 | 15 | 8.25 |
| 1000 | 25.0 | 3 | 0.002 | 238 | 8 | 8.21 |
| 1000 | 25.0 | 4 | 0.001 | 228 | 7 | 8.21 |
| 1000 | 25.0 | 5 | 0.001 | 223 | 12 | 8.18 |
| 500 | 12.5 | 1 | 0.031 | 383 | 13 | 8.34 |
| 500 | 12.5 | 2 | 0.005 | 314 | 9 | 8.21 |
| 500 | 12.5 | 3 | 0.001 | 287 | 5 | 8.13 |
| 500 | 12.5 | 4 | 0.001 | 278 | 4 | 8.15 |
| 500 | 12.5 | 5 | 0.002 | 257 | 3 | 8.15 |

2.5 60° C. Study

TABLE 9
Investigation of emulsion parameters
after preparation of an emulsion at 60° C.
(test temperature) and different homogenization pressures
T_{H} = 60° C.

| | | | | Mean | Micro- | |
|---|---|---|---|---|---|---|
| | Theoretical | | | particle | graph | |
| | temperature | | | diameter | [Number | |
| Pressure | increase | Passes | pFat5 | (PCS) | of | |
| [bar] | [° C.] | PSI-40 | [%] | [nm] | droplets] | pH |
| 1900 | 47.5 | 1 | 0.227 | 254 | 52 | 8.21 |
| 1900 | 47.5 | 2 | 0.069 | 219 | 50 | 8.15 |
| 1900 | 47.5 | 3 | 0.057 | 202 | 45 | 8.08 |
| 1900 | 47.5 | 4 | 0.048 | 199 | 30 | 8.08 |
| 1900 | 47.5 | 5 | 0.035 | 195 | 25 | 8.06 |
| 1500 | 37.5 | 1 | 0.124 | 265 | 27 | 8.17 |
| 1500 | 37.5 | 2 | 0.045 | 226 | 36 | 8.03 |
| 1500 | 37.5 | 3 | 0.029 | 208 | 20 | 8.05 |
| 1500 | 37.5 | 4 | 0.024 | 202 | 22 | 8.04 |
| 1500 | 37.5 | 5 | 0.023 | 201 | 25 | 7.97 |
| 1000 | 25.0 | 1 | 0.058 | 297 | 20 | 8.16 |
| 1000 | 25.0 | 2 | 0.011 | 253 | 13 | 8.10 |
| 1000 | 25.0 | 3 | 0.006 | 237 | 8 | 8.02 |
| 1000 | 25.0 | 4 | 0.004 | 225 | 6 | 8.01 |
| 1000 | 25.0 | 5 | 0.002 | 215 | 4 | 7.99 |
| 500 | 12.5 | 1 | 0.044 | 398 | 13 | 8.21 |
| 500 | 12.5 | 2 | 0.003 | 299 | 7 | 8.09 |
| 500 | 12.5 | 3 | 0.002 | 274 | 4 | 8.06 |
| 500 | 12.5 | 4 | 0.002 | 229 | 3 | 8.02 |
| 500 | 12.5 | 5 | 0.001 | 251 | 3 | 8.01 |

2.6 70° C. Study

TABLE 10
Investigation of emulsion parameters
after preparation of an emulsion at 70° C.
(test temperature) and different homogenization pressures
T_{H} = 70° C.

| | | | | Mean | Micro- | |
|---|---|---|---|---|---|---|
| | Theoretical | | | particle | graph | |
| | temperature | | | diameter | [Number | |
| Pressure | increase | Passes | pFat5 | (PCS) | of | |
| [bar] | [° C.] | PSI-40 | [%] | [nm] | droplets] | pH |
| 1900 | 47.5 | 1 | 0.190 | 252 | 72 | 8.14 |
| 1900 | 47.5 | 2 | 0.055 | 212 | 50 | 8.06 |
| 1900 | 47.5 | 3 | 0.033 | 207 | 36 | 8.01 |
| 1900 | 47.5 | 4 | 0.029 | 204 | 31 | 8.02 |
| 1900 | 47.5 | 5 | 0.020 | 203 | 50 | 8.03 |
| 1500 | 37.5 | 1 | 0.150 | 264 | 50 | 8.10 |
| 1500 | 37.5 | 2 | 0.118 | 222 | 55 | 8.01 |
| 1500 | 37.5 | 3 | 0.054 | 210 | 49 | 7.99 |
| 1500 | 37.5 | 4 | 0.053 | 202 | 54 | 7.98 |
| 1500 | 37.5 | 5 | 0.035 | 199 | 26 | 7.99 |
| 1000 | 25.0 | 1 | 0.145 | 293 | 53 | 8.11 |
| 1000 | 25.0 | 2 | 0.041 | 249 | 23 | 8.00 |
| 1000 | 25.0 | 3 | 0.012 | 229 | 13 | 7.92 |
| 1000 | 25.0 | 4 | 0.006 | 216 | 14 | 7.93 |
| 1000 | 25.0 | 5 | 0.009 | 206 | 16 | 7.95 |
| 500 | 12.5 | 1 | 0.981 | 378 | 68 | 8.20 |
| 500 | 12.5 | 2 | 0.008 | 291 | 4 | 8.10 |
| 500 | 12.5 | 3 | 0.004 | 268 | 4 | 8.02 |
| 500 | 12.5 | 4 | 0.001 | 248 | 2 | 8.00 |
| 500 | 12.5 | 5 | 0.002 | 242 | 3 | 7.97 |

The test results shown in Tables 5 to 10 show that the minimum medical standard required for parenteral administration of O/W emulsions, according to which the mean droplet diameter of the O/W emulsions should not exceed a value of 500 nm, is met by all the O/W emulsions produced. Furthermore, the results shown in tabular form in Tables 5 to 10 show that the mean droplet diameter can be reduced with increasing pressure and/or with increasing number of homogenization cycles.

In addition, the test results obtained show that droplets having a diameter, in particular a mean diameter, above 1 μm, in particular between 1 μm and 5 μm, preferably at a homogenizing pressure below 1000 bar, in particular at a homogenizing pressure of 500 bar, are comminuted. This makes it possible, particularly in the case of two counter-jet dispersers connected in series, to control the mean droplet diameter of the O/W emulsions to be produced via the first counter-jet disperser and the PFAT5 value of the O/W emulsions to be produced by the second, i.e. downstream, counter-jet disperser. In this manner, both the existing minimum standard with respect to the mean droplet diameter and the minimum standard required with respect to the PFAT5 value can be met in a targeted manner and the process quality can therefore be significantly increased.

3. Preparation of O/W Emulsions by Means of Homogenization at Different Pressure Levels

The fat emulsion prepared according to 1. was produced using two counter-jet dispersers (each of the PSI-40 type) connected in series. The results obtained here are shown in Tables 11 to 13 below.

2.7 First Pressure Stage 1900 Bar

TABLE 11
Investigation of the emulsion droplet diameter after preparation
of an emulsion using two different microfluidizer pressures

| | | | 1st pass, mean | 2nd pass, mean |
|---|---|---|---|---|
| Temperature | 1st Pass PSI-40 | 2nd Pass PSI-40 | particle diameter | particle diameter |
| [° C.] | pressure [bar] | pressure [bar] | (PCS) [nm] | (PCS) [nm] |
| 20 | 1900 | 1500 | 279 | 234 |
| 30 | 1900 | 1500 | 277 | 241 |
| 40 | 1900 | 1500 | 266 | 234 |
| 50 | 1900 | 1500 | 259 | 227 |
| 60 | 1900 | 1500 | 251 | 223 |
| 70 | 1900 | 1500 | 251 | 223 |
| 20 | 1900 | 1000 | 279 | 254 |
| 30 | 1900 | 1000 | 277 | 250 |
| 40 | 1900 | 1000 | 266 | 242 |
| 50 | 1900 | 1000 | 259 | 238 |
| 60 | 1900 | 1000 | 251 | 227 |
| 70 | 1900 | 1000 | 251 | 228 |
| 20 | 1900 | 500 | 279 | 274 |
| 30 | 1900 | 500 | 277 | 265 |
| 40 | 1900 | 500 | 266 | 266 |
| 50 | 1900 | 500 | 259 | 255 |
| 60 | 1900 | 500 | 251 | 220 |
| 70 | 1900 | 500 | 251 | 238 |

2.8 First Pressure Stage 1500 Bar

TABLE 12
Investigation of the emulsion droplet diameter after preparation
of an emulsion using two different microfluidizer pressures

| | | | 1st pass, mean | 2nd pass, mean |
|---|---|---|---|---|
| Temperature | 1st Pass PSI-40 | 2nd Pass PSI-40 | particle diameter | particle diameter |
| [° C.] | pressure [bar] | pressure [bar] | (PCS) [nm] | (PCS) [nm] |
| 20 | 1500 | 1000 | 298 | 239 |
| 30 | 1500 | 1000 | 298 | 259 |
| 40 | 1500 | 1000 | 280 | 257 |
| 50 | 1500 | 1000 | 271 | 246 |
| 60 | 1500 | 1000 | 267 | 238 |
| 70 | 1500 | 1000 | 268 | 233 |
| 20 | 1500 | 500 | 298 | 276 |
| 30 | 1500 | 500 | 298 | 270 |
| 40 | 1500 | 500 | 280 | 269 |
| 50 | 1500 | 500 | 271 | 254 |
| 60 | 1500 | 500 | 267 | 256 |
| 70 | 1500 | 500 | 268 | 251 |

2.9 First Pressure Stage 1000 Bar

TABLE 13
Investigation of the emulsion droplet diameter after preparation
of an emulsion using two different microfluidizer pressures

| | | | 1st pass, mean | 2nd pass, mean |
|---|---|---|---|---|
| Temperature | 1st Pass PSI-40 | 2nd Pass PSI-40 | particle diameter | particle diameter |
| [° C.] | pressure [bar] | pressure [bar] | (PCS) [nm] | (PCS) [nm] |
| 20 | 1000 | 500 | 367 | 333 |
| 30 | 1000 | 500 | 329 | 304 |
| 40 | 1000 | 500 | 317 | 306 |
| 50 | 1000 | 500 | 318 | 282 |
| 60 | 1000 | 500 | 295 | 267 |
| 70 | 1000 | 500 | 287 | 265 |

The results presented in tabular form show that the medical minimum standard required for parenterally administered O/W emulsions with respect to the mean droplet diameter is met by all the O/W emulsions produced. The results also show that the mean droplet diameter can be further reduced by using a second counter-jet disperser connected in series. If the second counter-jet disperser is also operated at a homogenizing pressure (pump pressure)<1000 bar, in particular at a homogenizing pressure of 500 bar, the PFAT5 value applicable to parenterally administered O/W emulsions can be significantly undercut. Overall, therefore, a significant increase in the process quality can be achieved, particularly with regard to the mean droplet diameter and the PFAT5 value of the O/W emulsions to be produced.

1. A method for producing an O/W emulsion comprising the following steps:
a) providing an oil phase and a water phase;
b) premixing the oil phase and the water phase to form an O/W pre-emulsion; and
c) homogenizing the O/W pre-emulsion to form an O/W emulsion by at least one counter-jet disperser.

2. The method according to claim 1, wherein step b) is carried out by at least one rotor-stator disperser.

3. The method according to claim 2, wherein the oil phase and the water phase are fed to the at least one rotor-stator disperser spatially separated from each other.

4. The method according to claim 2, wherein the oil phase and the water phase are fed to the at least one rotor-stator disperser by a tube-in-tube arrangement.

5. The method according to claim 2, wherein the oil phase and the water phase are passed through a droplet comminution zone of the at least one rotor-stator disperser.

6. The method according to claim 1, wherein step c) is carried out by a pump pressure of 1000 bar to 1900 bar.

7. The method according to claim 1, wherein step c) is carried out at a temperature of the O/W pre-emulsion of 30° C. to 80° C.

8. The method according to claim 1, wherein the O/W pre-emulsion is passed repeatedly through the at least one counter-jet disperser when carrying out step c).

9. The method according to claim 1, wherein step c) is carried out by a plurality of counter-jet dispersers.

10. The method according to claim 1, wherein step c) is carried out by a first counter-jet disperser and a second counter-jet disperser connected in series.

11. The method according to claim 10, wherein the first counter-jet disperser is operated at a higher pump pressure than the second counter-jet disperser.

12. The method according to claim 10, wherein the first counter-jet disperser is operated at a pump pressure of at most 1500 bar.

13. The method according to claim 1, wherein a pressure reducer is connected downstream of the at least one counter-jet disperser.

14. An O/W emulsion, produced or producible according to the method of claim 1 and/or having a PFAT5 value <0.04%.

15. A system for producing an O/W emulsion according to the method of claim 1, wherein the system has at least one disperser for premixing an oil phase and a water phase to form an O/W pre-emulsion and comprises the at least one counter-jet disperser for homogenizing the O/W pre-emulsion to an O/W emulsion.
